(19) United States
(12) Patent Application Publication
(10) Pub. No.: US 2024/0423483 A1
(43) Pub. Date: Dec. 26, 2024

(54) NON-INVASIVE HEMODYNAMIC MONITORING SYSTEM

(71) Applicant: SILVERLEAF MEDICAL SCIENCES INC., Chino, CA (US)

(72) Inventors: Jianwei (Arnold) Zheng, Chino, CA (US); Islam Abudayyeh, Redlands, CA (US); Wenzan Zhang, Irvine, CA (US)

(73) Assignee: SILVERLEAF MEDICAL SCIENCES INC., Chino, CA (US)

(21) Appl. No.: 18/212,073

(22) Filed: Jun. 20, 2023

(51) Int. Cl. A61B 5/0205; A61B 5/1455; A61B 5/33; G16H 40/63

(52) U.S. Cl. CPC A61B 5/0205; A61B 5/1455; A61B 5/33; G16H 40/63

(57) ABSTRACT

The present invention relates to a portable non-invasive hemodynamic monitoring system that obtains various parameters related to cardiovascular function, including blood oxygen saturation, perfusion index, pulse wave, electrocardiogram, respiration, bioimpedance, etc., and analyzes the collected data to obtain parameter data such as cardiac output, non-invasive blood pressure, cardiac index, pulmonary arterial pressure (PAP), etc.

### BACKGROUND

Hemodynamic monitoring of patients is important in ICU, especially those with acute left ventricular insufficiency and heart failure. Through the quantitative, dynamic, and continuous monitoring of hemodynamic parameters, regular analysis can be carried out, and the patient's condition and the efficacy of clinical treatment can be judged.

At present, most hospitals use invasive methods to obtain hemodynamic parameters of patients with a cardiovascular symptom. Several parameters essential for hemodynamic monitoring, such as pulmonary arterial pressure (PAP), pulmonary arterial widget pressure (PAWP), and central venous pressure (CVP) are typically only reliably measured by invasive means. Arterial blood pressure (ABP) can be measured by intra-arterial catheters. Some noninvasive methods can estimate ABP, but catheter detection is more reliable and accurate. The measurement of SV and CO have a similar situation as ABP. Due to the high cost, high risk, few indications, and limited access to parameters, it is limited to the detection of critically ill patients, when necessary, seriously restricting the effective treatment of the disease.

Non-invasive hemodynamic monitoring can quickly provide the hemodynamic basis for clinical diagnosis by virtue of its non-invasive risk of infection, and it has few non-invasive complications, less pain for patients, easy to accept and implement, and many other advantages. However, non-invasive hemodynamic monitoring is not readily available for the above-mentioned essential parameters. Thus, there is a need in the field to replace invasive hemodynamic monitoring with non-invasive monitoring for the management of cardiovascular diseases.

### SUMMARY OF THE INVENTION

In one aspect, the present invention provides a non-invasive and portable device for monitoring hemodynamics, which comprises
a data acquisition unit comprising
   a host machine;
   a body surface lead; and
   a photoelectric earplug;
wherein the host machine comprises a signal interface and a data collection and conversion module; wherein one end of the body surface lead is configured to connect to an electrode or electrode pad attached to the body surface of a person in need and the other end of the body surface lead is configured to connect with the lead interface located in the host machine; wherein the data collection and conversion module comprises a pulse oximetry module; and wherein the photoelectric earplug comprises a reflective photoelectric sensor.

In one embodiment, the data collection and conversion module comprises at least an additional data collection and conversion module selected from a group consisting of a reflective photoelectric module, an electrocardiogram (ECG) module, a bioimpedance module, a motion module, a blood oxygen module, a perfusion index module, a pulse wave module, a respiration module, a blood pressure module, a body temperature module, an cardiac output module, a photoplethysmography module, a phonocardiogram module, an impedance cardio graphic module, a respiration impedance module, a peripheral venous pressure module, a peripheral arterial pressure module, and a combination thereof.

In a further embodiment, the additional data collection and conversion module is an electrocardiogram (ECG) module.

In a further embodiment, the electrocardiogram (ECG) module comprises a combined respiration module (an ECG and respiration module).

In a further embodiment, the additional data collection and conversion module is an impedance detection module.

In a further embodiment, the additional data collection and conversion module is a motion monitoring module.

In another embodiment, the host machine comprises a digital signal processing module.

In a further embodiment, the host machine comprises supporting modules including a data storage module, a data transmission module, a power management module, and a rechargeable battery and charging module.

In a further embodiment, the digital signal processing module is configured to connect with the data collection and conversion modules and with the supporting modules to control said modules and process and transmit data among said modules.

In another embodiment, the non-invasive and portable device comprises a data analysis unit, wherein the data analysis unit comprises a computational hardware and a mathematical algorithm to process and analyze data collected by the host machine. The data analysis unit and the host machine are connected in wired or wireless manner.

In a further embodiment, the computational hardware comprises a computer, a mobile device, a cloud server, or a combination thereof.

In another aspect, the present invention provides a non-invasive and portable device for monitoring hemodynamics, which comprises
a data acquisition unit comprising:
   a host machine comprising:
      a signal interface; and
      a data collection and conversion module;
   a body surface lead;
   a photoelectric earplug comprising a reflective photoelectric sensor;
a data analysis unit comprising a computational hardware and a mathematical algorithm;
wherein one end of the body surface lead is configured to connect to an electrode or electrode pad attached to the body surface of a person in need and the other end of the body surface lead is connected with the lead interface located in the host machine; wherein the data collection and conversion module comprises a pulse oximetry module; and wherein the computational hardware comprises a computer, a mobile device, a cloud server, or a combination thereof.

In one embodiment, the data collection and conversion module comprises at least an additional data collection and conversion module selected from a group consisting of a reflective photoelectric module, an electrocardiogram (ECG) module, a bioimpedance module, a motion module, a blood oxygen module, a perfusion index module, a pulse wave module, a respiration module, a blood pressure module, a body temperature module, a cardiac output module, a photoplethysmography module, a phonocardiogram module, an impedance cardio graphic module, a respiration impedance module, a peripheral venous pressure module, a peripheral arterial pressure module, and a combination thereof.

In a further embodiment, the additional data collection and conversion module is an electrocardiogram (ECG) module.

In another further embodiment, the electrocardiogram (ECG) module comprises a combined respiration module (an ECG and respiration module).

In a further embodiment, the additional data collection and conversion module is an impedance detection module.

In another embodiment, the additional data collection and conversion module is a motion monitoring module.

In another embodiment, the host machine comprise supporting modules including a data storage module, a data transmission module, a power management module, and a rechargeable battery and charging module.

In a further embodiment, the data analysis unit is connected, in wired or wireless manner, with the data collection and conversion modules of the host machine and with the supporting modules of the host machine to control said modules, transmit data among said modules, and process and analyze the data collected by the host machine.

### BRIEF DESCRIPTION OF DRAWINGS

FIG. 1 illustrates a non-invasive and portable device for monitoring hemodynamics in accordance with one implementation of the present disclosure.

FIG. 2 illustrates a wearing method of ECG leads and impedance detection leads connected by flexible wires and connections between the body surface leads and the host machine.

FIG. 3 illustrates a wearing method of ECG lead, where the ECG lead adopts an integrated sheet structure. In this embodiment, the host machine and the ECG lead sheet are connected through a fixed interface according to the attachment position.

FIG. 4 illustrates the positions of the data cable attached to photoelectric earplugs.

FIG. 5 illustrates, in one embodiment, a reflective photoelectric sensor and its circuit enclosed inside photoelectric earplugs.

### DETAILED DESCRIPTION OF INVENTION

Non-invasive hemodynamic monitoring uses a method without mechanical damage to the body, through the skin or mucous membranes, etc. Non-invasive hemodynamic monitoring is efficient, low-cost, risk-free, and has no contraindications to obtain as many hemodynamic parameters related to cardiovascular functions as possible. Non-invasive detection technology may be used not only for examination and treatment of critically ill patients, but also for synchronous monitoring of pulse waves and electrocardiogram. Non-invasive detection technology can detect fatal lesions early, and can be used for early diagnosis, disease classification, medication guidance, and prognosis evaluation for general patients. Because many diseases of the cardiovascular system have changed many hemodynamic parameters before patients have obvious symptoms, this technology can also be used for physical examination of people with a high risk of cardiovascular disease, which is conducive to the early detection and treatment. Non-invasive detection technology can also be used for the implementation of preoperative and postoperative cardiac function determination of patients undergoing coronary revascularization. It may reduce or eliminate the need for coronary angiography, reduce the pain of patients and reduce the cost of treatment. In clinical research and treatment, non-invasive detection technology can easily obtain objective data on treatment effects and is beneficial to the improvement of the treatment plan.

Examples of non-invasive hemodynamic monitoring include heart rate (HR), non-invasive blood pressure (NIBP), pulse oxygen saturation (SpO2), and cardiac output (CO). The ideal non-invasive hemodynamic monitoring system should have certain accuracy, provide information similar to trauma (invasive) monitoring, can display physiological data continuously and synchronously, have no or few complications, and present high sensitivity suitable for early diagnosis.

Most of the currently used collection methods of hemodynamic products are invasive or minimally invasive, having several obvious disadvantages, such as known risks of complications or infection, pain experienced by patients, high cost, and time-wasting. Furthermore, most currently available non-invasive monitoring equipment cannot achieve continuous dynamic monitoring. For example, ECG alone cannot fully evaluate the overall health of the heart. Holter and various remote ECG monitors cannot complete hemodynamic monitoring. For blood oxygen pulse monitoring, the transmission type blood oxygen saturation detection technology is used. The light-emitting tube and photosensitive sensor are placed on both sides of the human body and the sensor is kept in vertical position. Therefore, the test position is mostly selected on the limbs. The end of the sensor, such as the finger, toe end, or earlobe, cannot detect the blood oxygen saturation of multiple parts of the human body. Secondly, the transmission type mostly adopts clip-type or sleeve-type measurement, which causes users to have oppressive feeling when wearing it for a prolonged period of time. Moreover, such transmission type affects blood circulation, causes discomfort and measurement errors, and has certain limitations in application. In addition, most non-invasive monitoring devices are bulky and cannot be carried around.

In one aspect, the instant disclosure provides a non-invasive, portable, and comfortable hemodynamic monitoring device, which can dynamically and continuously monitor and obtain information about the cardiovascular function without causing mechanical or physical damage to the body. It collects various parameters, including for example, blood oxygen saturation, perfusion index, pulse wave, ECG, respiration, blood pressure, bioimpedance, cardiac output, etc., and analyzes the collected data to obtain other parameters related to cardiovascular functions.

In one embodiment, the non-invasive portable device includes a data acquisition unit and a data analysis unit.

### A. Data Acquisition Unit

In a further embodiment, the data acquisition unit comprises a host machine, body surface leads, and photoelectric earplugs.

### Host Machine

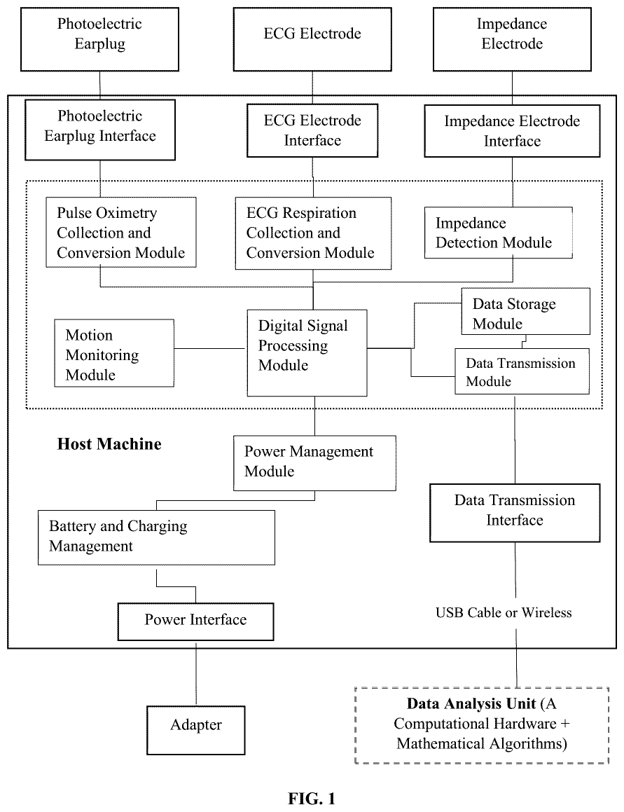

In one embodiment, the host machine (FIG. 1) comprises one or more signal interfaces for each corresponding body surface lead, one or more data collection and conversion modules, optionally one or more digital signal processing modules, and optionally one or more supporting modules. The signal interfaces are either configured as independent structures separate from the corresponding data collection and conversion modules or integrated partially or fully into the corresponding data collection and conversion modules.

Examples of the data collection and conversion modules of host machine may include pulse oximetry module, reflective photoelectric module, electrocardiogram (ECG) module, bioimpedance module, motion module, blood oxygen module, perfusion index module, pulse wave module, respiration module, blood pressure module, body temperature module, and cardiac output module, photoplethysmography module, phonocardiogram module, peripheral arterial pressure module, impedance cardio graphic module, respiration impedance module, peripheral venous pressure module, or peripheral arterial pressure module.

The host machine may contain one or more digital signal processing modules, which are configured to work independently from the external computer or mobile device. Alternatively, the digital signal processing module can be integrated into the external computer or mobile device. In certain embodiments, both host machine and the external computer or mobile contain one or more one or more digital signal processing modules. The digital signal processing modules located in the host machine can work independently or work collaboratively with the digital signal processing modules integrated in the computer or mobile device.

Examples of the supporting modules include a data storage module, a data transmission module (configured to transmit data to a control device, computer, mobile device, or a cloud server), a power management module, a battery, or a charging module.

As to the data collection and conversion modules, the host machine can comprise a pulse oximetry collection and conversion module. The pulse oximetry collection and conversion module (pulse oximetry module) has the functions of controlling the light source and signal conversion and converts the electrical signal collected by the photoelectric earplugs into digital signals, so as to obtain the photoplethysmogram (PPG) and calculate the perfusion index of blood oxygen.

The host machine can comprise an electrocardiogramespiration signal collection and conversion module (ECG and respiration module). The ECG and respiration module receives electrocardiogramaignals and respiratory waves collected by the electrocardiogramapiration body surface electrodes and converts them into digital signals.

The host machine can contain an impedance detection module. The impedance detection module sends and receives stimulus analog electrical signals through impedance detection body surface electrodes, and then converts them into digital signals.

Another suitable data collection and conversion module for the host machine is a motion monitoring module. The motion monitoring module is configured to monitor the motion state of the subject and identify motion interference.

The host machine can contain a digital signal processing module, which is connected to other modules. The digital signal processing module is configured to control other modules, process and transmit data of each module.

In one embodiment, the supporting modules of the host machine comprises at least one module selected from a data storage module, a data transmission module, a power management module, a battery and charging module, or a combination thereof.

The storage module stores pulse wave data, electrocardiogram data, respiration wave, etc. according to a certain sampling rate, which facilitates further analysis and processing after the data is uniformly read.

The data transmission module is responsible for transportation of the measured information, which can be wired or wireless. The transmitted data includes real-time data and user history records. The data receiving end is a computer or other smart device.

The power management module converts battery power into power required by each module. The battery and the charging module includes a rechargeable lithium battery and a charging circuit. The host machine can be charged through an adapter and a charging cable

In a preferred embodiment, the host machine comprises a pulse oximetry collection and conversion module (pulse oximetry module), an electrocardiogramaspiration signal collection and conversion module (ECG and respiration module), an impedance detection module, a motion monitoring module, and a digital signal processing module.

The pulse oximetry module comprises a main chip of AFE4404 from Texas Instruments and an analog front end for optical biosensing applications, which includes a transmit path and a receive path. The transmission path includes the LED driver and the circuit of driving current selection, which controls the LED to make the red light and the infrared light source emit light alternately at the specified current intensity. Part of the emitted light will be absorbed and transmitted, and the other part will be reflected back to the receiving tube. The receiving tube generates a current signal, and then the current signal is converted into a digital signal through the programmable trans-impedance amplifier, ambient light elimination circuit, analog-to-digital converter in the receiving path, and then output to the digital signal processing module through an I2C interface. After filter processing, the pulse wave was obtained, and the blood oxygen saturation and perfusion index were calculated.

The ECG and respiration module comprises a main chip of AFE1292R from Texas Instruments, which is a multi-channel synchronous sampling 24-bit analog-to-digital converter and integrates ECG and respiratory impedance measurement functions.

The impedance detection module uses a four-electrode method. Specifically, a pair of stimulation electrodes are used to provide stimulus current, and a pair of acquisition electrodes are used to measure the voltage drop on the human body. This part can use a chip of AD5933 and adopt the pressure-current conversion design. It converts the excitation voltage signal sent by AD5933 into an AC constant current signal and pass the multi-frequency constant current raised to 2.5V through the excitation electrode and pass through the human body. The voltage generated on the human body is sent to the analog-to-digital conversion circuit for conversion processing and impedance characteristic calculation through a differential amplifier with high input impedance and high common mode rejection ratio to ensure the accuracy of the measurement.

The motion monitoring module uses a 3-axis accelerometer in the design, because the collected signal is easily affected by human motion interference, which causes distortion of the measurement results. ADXL345 from ADI can be integrated into the module to obtain motion artifact noise for further processing.

In the same preferred embodiment, the digital signal processing module contains a STM32L151RC microcontroller to control other modules, process and transmit data of each module.

### Body Surface Leads

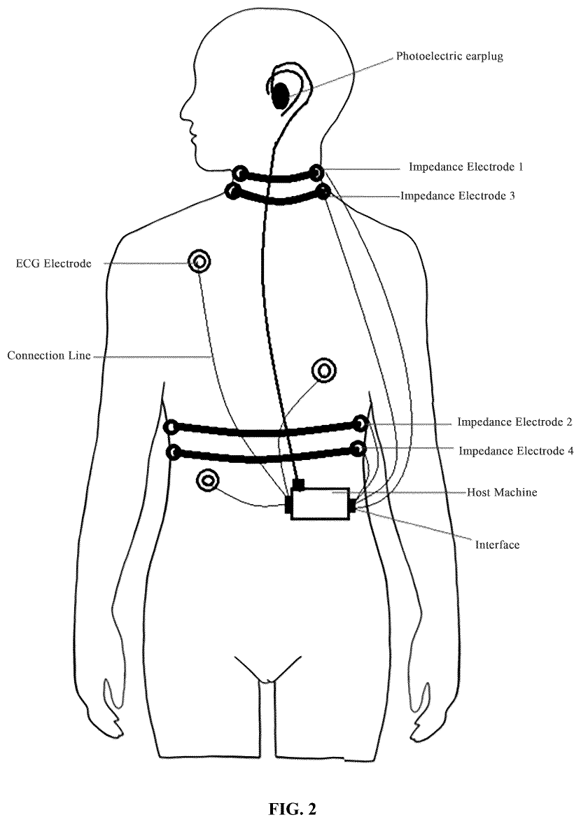

In the above preferred embodiment, the data acquisition unit comprises three groups of body surface leads, and each group has multiple leads: one set of ECG leads, one set of impedance detection excitation leads, and one set of impedance detection acquisition leads. (See FIG. 2).

One end of the lead is connected to the electrode pad (which can be a button-type connection) attached to the human body surface, and the attachment position is based on actual requirements. The other end is connected with a lead interface so that it can be inserted into the host machine. The lead can be of a flexible wire type, a flexible circuit type, an integrated chip type, or can also be worn on the subject in the form of a vest.

The ECG leads, which can contain multiple electrodes, are attached to the corresponding position of the wearer's chest skin after the electrode pads are buckled.

Impedance detection excitation leads include a pair of electrodes, electrode 1, electrode 2. The electrode 1 ring is attached to the neck of the human body, and the electrode 2 ring is attached to the lower chest of the human body. A set of impedance detection acquisition leads, including a pair of electrodes, electrode 3, electrode 4. The electrode 3 ring is attached to the neck of the human body, below the electrode 1, and the electrode 4 ring is attached to the lower chest of the human body, above the electrode 2.

The ECG lead and the impedance detection lead can have separate interfaces, which are connected to the host machine, respectively.

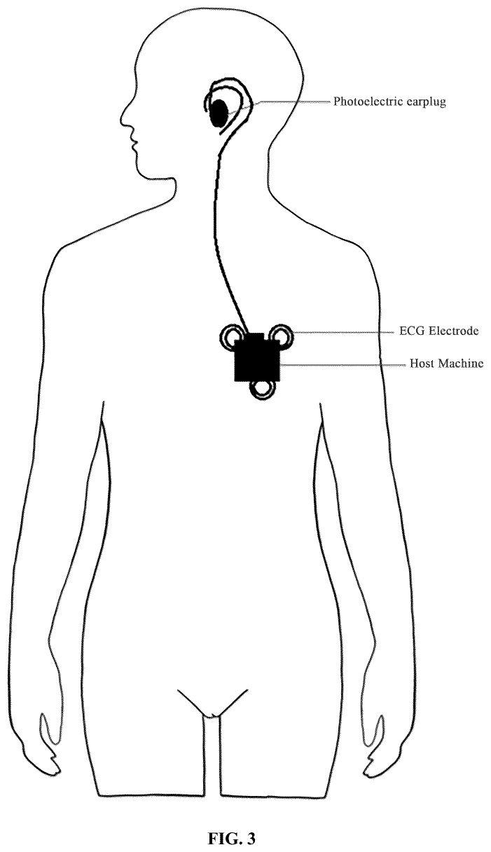

Whether the corresponding lead needs to be worn or the form of the lead can be selected according to the monitoring requirements. As shown in FIG. 2, it indicates a wearing method of ECG leads and impedance detection leads connected by flexible wires. The host machine is carried by the person in need and the position is flexible. As shown in FIG. 3, only the ECG lead is worn, and the ECG lead adopts an integrated sheet structure. The host machine and the ECG lead sheet are connected through a fixed interface according to the attachment position.

### Photoelectric Earplugs

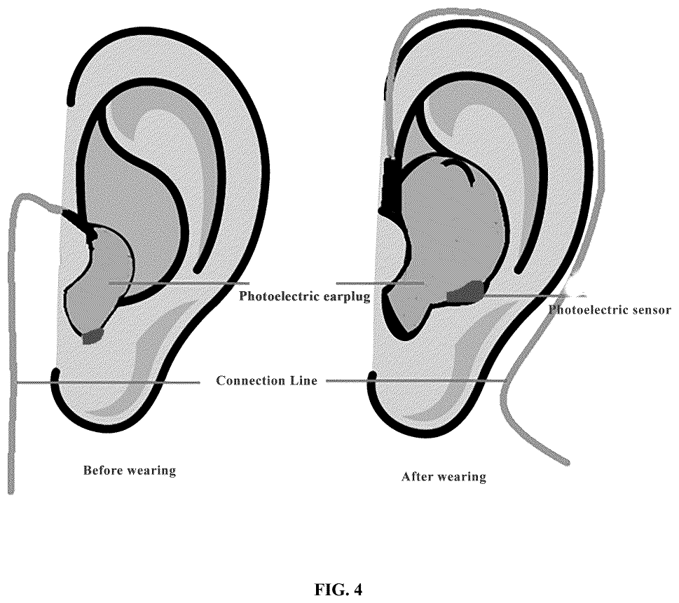

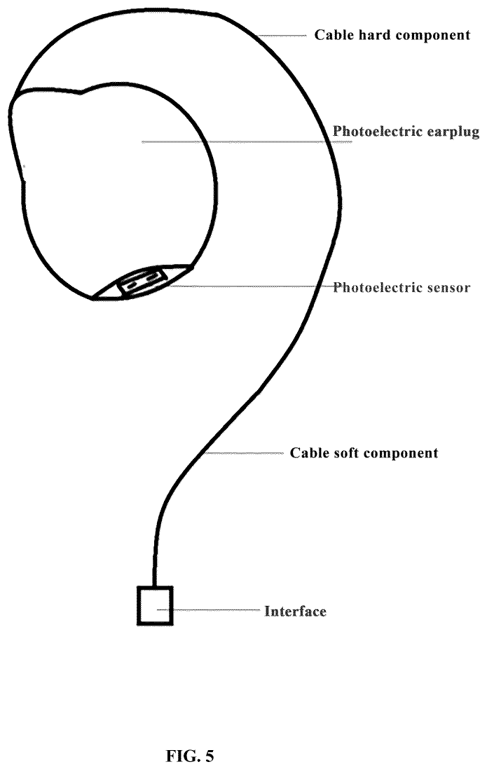

One end of the photoelectric earplug is an earplug structure, which is worn in the external auditory canal of the human body. Its structure should maintain its wearing stability and ensure that the photoelectric sensor firmly contacts with the skin of the external auditory canal. It can be made of silicone material. Since the texture of silicone is relatively soft, its shape can vary according to the structure of the external auditory canal. The adjustment space ensures the versatility and wearing comfort of the earplugs. The earplug part has a data cable connected to the other end, and the other end is equipped with a connection port, which is connected to the host machine. The part of the data cable close to the earplugs (5-10 cm line length) is hardened and shaped to make the data cable into an arc shape. After the earplugs are worn, the data cable can be fixed behind the ear to stabilize the earplugs (Shown in FIG. 4). The rest of the data cable can be a segment of soft wire. There is a reflective photoelectric sensor and its circuit inside the earplug (Shown in FIG. 5). The reflective sensor includes a light-emitting tube and at least one light-sensitive sensor. The light-emitting tube and the light-sensitive sensor are arranged on the same side of the measured part. Since the sensor volume is small, it is not affected by the space and is more suitable for fixing in earbuds. A small reflective photoelectric sensor, NJL5501R can be used, which integrates a red LED, an infrared LED, and a high-sensitivity photodiode in one package. The reflective photoelectric sensor is fixed inside the earplug, and a window is left on the surface of the earplug to expose the sensor. After the earplug is worn on the ear, the window is attached to the flat part of the external auditory canal, which can collect pulse wave data more comfortably and stably. It is suitable for long-term monitoring.

### B. Data Analysis Unit

In another embodiment, the data collected and processed by the digital signal processing module are transmitted through data transmission interface to a data analysis unit. The data analysis unit comprises a computational hardware, such as a computer, a mobile device, a cloud server, or other external device and mathematical algorithms installed on the computational hardware.

Here, a mobile device generally refers to a handheld portable device, which runs a mobile operating system and has sufficient computational power to run a software or mobile app. Typical mobile devices include smartphones and tablets.

The data may be further uploaded to a cloud server from the computer, the mobile device, or the other external device. Alternatively, the collected data may be uploaded directly to the cloud server from the digital signal processing module. The collected data is processed and analyzed by the data analysis unit through serial or parallel computation. The data analysis unit generates several essential hemodynamics parameters, such as pulmonary arterial pressure (PAP), to assist clinicians to monitor the progression of patient's cardiovascular disease and make treatment decisions when necessary. Further, the data transmission module in the present disclosure supports real-time display of data by the host computer or the mobile device in a wired or wireless manner.

Hereinafter, the present invention will be described in more detail by way of Examples. The invention will be more readily understood by reference to the following examples. However, the technical scope of the present invention is not intended to be limited only to the following Examples.

### III. Example. A Non-Invasive Portable Device for Monitoring Hemodynamic Parameters

A non-invasive portable device for monitoring patient's hemodynamic parameter was made to evaluate its functions. Specifically, the device comprises body surface leads, a host machine, and photoelectric earplugs.

The device has three groups of body surface leads, and each group has multiple leads: one set of ECG leads, one set of impedance detection excitation leads, and one set of impedance detection acquisition leads. (See FIG. 2).

One end of the lead is connected to the electrode pad (which can be a button-type connection) attached to the human body surface, and the attachment position is based on actual requirements. The other end is connected with a lead interface so that it can be inserted into the host machine. The lead can be of a flexible wire type, a flexible circuit type, an integrated chip type, or can also be worn on the subject in the form of a vest.

The ECG leads, which can contain multiple electrodes, are attached to the corresponding position of the wearer's chest skin after the electrode pads are buckled.

Impedance detection excitation leads include a pair of electrodes, electrode 1, electrode 2. The electrode 1 ring is attached to the neck of the human body, and the electrode 2 ring is attached to the lower chest of the human body. A set of impedance detection acquisition leads, including a pair of electrodes, electrode 3, electrode 4. The electrode 3 ring is attached to the neck of the human body, below the electrode 1, and the electrode 4 ring is attached to the lower chest of the human body, above the electrode 2.

The ECG lead and the impedance detection lead can have separate interfaces, which are connected to the host machine, respectively.

Whether the corresponding lead needs to be worn or the form of the lead can be selected according to the monitoring requirements. As shown in FIG. 2, it indicates a wearing method of ECG leads and impedance detection leads connected by flexible wires. The host machine is carried by a person in need and the position is flexible. As shown in FIG. 3, only the ECG lead is worn, and the ECG lead adopts an integrated sheet structure. The host machine and the ECG lead sheet are connected through a fixed interface according to the attachment position.

The host machine of the device contains three signal interfaces and three corresponding data collection and conversion modules. Specifically, the signal interfaces and data collection modules combinations used in the device are (1) photoelectric earplug interface—pulse oximetry collection and conversion module (as described above); (2) ECG electrode interface—ECG respiration collection and conversion module; and (3) impedance electrode interface—impedance detection module. The pulse oximetry module comprises a main chip of AFE4404 from Texas Instruments and an analog front end for optical biosensing applications, which includes a transmit path and a receive path. The ECG and respiration module comprises a main chip of AFE1292R from Texas Instruments, which is a multi-channel synchronous sampling 24-bit analog-to-digital converter and integrates ECG and respiratory impedance measurement functions. The impedance detection module uses a four-electrode method. Specifically, a pair of stimulation electrodes are used to provide stimulus current, and a pair of acquisition electrodes are used to measure the voltage drop on the human body. The impedance detection module contains a chip of AD5933 and adopt the pressure-current conversion design.

The host machine further contains a motion monitoring module, which uses a 3-axis accelerometer in the design. ADXL345 from ADI can be integrated into the module to obtain motion artifact noise for further processing.

In addition, the host machine contains a digital signal processing module having a STM32L151RC microcontroller to control other modules, process and transmit data of each module.

The host machine further comprises supporting modules such as a data storage module, a data transmission module, a power management module, and a battery and charging module.

The configurations and connections among the above-mentioned components are illustrated in FIG. 1.

The device is further connected to a data analysis unit. The data analysis unit comprises a computational hardware, such as a computer, a mobile device, or a cloud server and mathematical algorithms installed on the computational hardware. The data analysis unit is responsible for processing the data collected by the device and calculation of essential hemodynamics parameters, such as pulmonary arterial pressure (PAP), to assist clinicians to monitor the progression of patient's cardiovascular disease and make treatment decisions when necessary.

The device described in this example is suitable for long-term continuous monitoring. When measuring blood oxygen, perfusion index, and pulse wave, there is no need to clamp the peripheral parts of the human body, and only the photoelectric earplugs need to be placed in the external auditory canal, which completely solves the discomfort caused by the clip-type acquisition method and the inconvenience brought to movements. It can non-invasively monitor and record the user's ECG, respiration, pulse, blood oxygen, perfusion index, impedance, and other data throughout the process. The data collected by the device can also be uploaded to a PC through the USB cable, and the data can be read through the host computer software. After integrated analysis and processing, more blood flow such as cardiac output, non-invasive blood pressure, and cardiac index can be obtained. Furthermore, the data transmission module in the present device supports real-time display of data by the host computer, which can be the laptop computer or an intelligent wireless device connected in a wired or wireless manner.

What is claimed is:

1. A non-invasive and portable device for monitoring hemodynamics, comprising:
a data acquisition unit comprising:
a host machine;
a body surface lead; and
a photoelectric earplug;
wherein the host machine comprises a signal interface and a data collection and conversion module;
wherein one end of the body surface lead is configured to connect to an electrode or electrode pad attached to the body surface of a person in need and the other end of the body surface lead is configured to connect with the lead interface located in the host machine;
wherein the data collection and conversion module comprises a pulse oximetry module; and
wherein the photoelectric earplug comprises a reflective photoelectric sensor.

2. The non-invasive and portable device of claim 1, wherein the data collection and conversion module comprises at least an additional data collection and conversion module selected from a group consisting of a reflective photoelectric module, an electrocardiogram (ECG) module, a bioimpedance module, a motion module, a blood oxygen module, a perfusion index module, a pulse wave module, a respiration module, a blood pressure module, a body temperature module, an cardiac output module, a photoplethysmography module, a phonocardiogram module, an impedance cardio graphic module, a respiration impedance module, a peripheral venous pressure module, a peripheral arterial pressure module, and a combination thereof.

3. The non-invasive and portable device of claim 2, wherein the additional data collection and conversion module is an electrocardiogram (ECG) module.

4. The non-invasive and portable device of claim 3, wherein the electrocardiogram (ECG) module comprises a combined respiration module (an ECG and respiration module).

5. The non-invasive and portable device of claim 2, wherein the additional data collection and conversion module is an impedance detection module.

6. The non-invasive and portable device of claim 2, wherein the additional data collection and conversion module is a motion monitoring module.

7. The non-invasive and portable device of claim 1, wherein the host machine comprises a digital signal processing module.

8. The non-invasive and portable device of claim 7, wherein the host machine comprises supporting modules including a data storage module, a data transmission module, a power management module, and a rechargeable battery and charging module.

9. The non-invasive and portable device of claim 8, wherein the digital signal processing module is configured to connect with the data collection and conversion modules and with the supporting modules to control said modules and process and transmit data among said modules.

10. The non-invasive and portable device of claim 8, further comprising a data analysis unit, wherein the data analysis unit comprises a computational hardware and a mathematical algorithm to process and analyze data collected by the host machine.

11. The non-invasive and portable device of claim 8, wherein the data analysis unit and the host machine are connected in wired or wireless manner.

12. The non-invasive and portable device of claim 8, wherein the computational hardware comprises a computer, a mobile device, a cloud server, or a combination thereof.

13. A non-invasive and portable device for monitoring hemodynamics, comprising:
a data acquisition unit comprising:
a host machine comprising:
a signal interface; and
a data collection and conversion module;
a body surface lead;
a photoelectric earplug comprising a reflective photoelectric sensor;
a data analysis unit comprising a computational hardware and a mathematical algorithm;
wherein one end of the body surface lead is configured to connect to an electrode or electrode pad attached to the body surface of a person in need and the other end of the body surface lead is connected with the lead interface located in the host machine;
wherein the data collection and conversion module comprises a pulse oximetry module; and
wherein the computational hardware comprises a computer, a mobile device, a cloud server, or a combination thereof.

14. The non-invasive and portable device of claim 13, wherein the data collection and conversion module comprises at least an additional data collection and conversion module selected from a group consisting of a reflective photoelectric module, an electrocardiogram (ECG) module, a bioimpedance module, a motion module, a blood oxygen module, a perfusion index module, a pulse wave module, a respiration module, a blood pressure module, a body temperature module, an cardiac output module, a photoplethysmography module, a phonocardiogram module, an impedance cardio graphic module, a respiration impedance module, a peripheral venous pressure module, a peripheral arterial pressure module, and a combination thereof.

15. The non-invasive and portable device of claim 14, wherein the additional data collection and conversion module is an electrocardiogram (ECG) module.

16. The non-invasive and portable device of claim 15, wherein the electrocardiogram (ECG) module comprises a combined respiration module (an ECG and respiration module).

17. The non-invasive and portable device of claim 14, wherein the additional data collection and conversion module is an impedance detection module.

18. The non-invasive and portable device of claim 14, wherein the additional data collection and conversion module is a motion monitoring module.

19. The non-invasive and portable device of claim 13, wherein the host machine comprise supporting modules including a data storage module, a data transmission module, a power management module, and a rechargeable battery and charging module.

20. The non-invasive and portable device of claim 13, wherein the data analysis unit is connected, in wired or wireless manner, with the data collection and conversion modules of the host machine and with the supporting modules of the host machine to control said modules, transmit data among said modules, and process and analyze the data collected by the host machine.
